(19) United States
(12) Patent Application Publication
(10) Pub. No.: US 2018/0256923 A1
(43) Pub. Date: Sep. 13, 2018

(54) SYSTEMS AND KITS FOR USER CUSTOMIZABLE COSMETICS, AND METHODS FOR THE PREPARATION OF USER CUSTOMIZED COSMETICS

(71) Applicant: Vanessa Ungvarsky, Easton, PA (US)

(72) Inventor: Vanessa Ungvarsky, Easton, PA (US)

(21) Appl. No.: 15/916,539

(22) Filed: Mar. 9, 2018

Related U.S. Application Data
(60) Provisional application No. 62/469,162, filed on Mar. 9, 2017.

(51) Int. Cl. A61Q 3/02; A61K 8/18

(52) U.S. Cl. CPC A61Q 3/02; A61K 2800/87; A45D 2200/25; A61K 8/18

(57) ABSTRACT

Systems, kits and methods for client customizable cosmetics including but not limited to foundation, blush, eye-shadow, lip products, and nail polish. Systems, kits and methods include a full range of on demand mixing powders and solutions, along with tools for creating, mixing, naming, labeling, and tracking past use for future reference. Systems, kits and methods can be provided in larger packages or in custom, fit to order amounts for users. Systems, kits and methods provide basic, sometimes raw materials, so clients can create cosmetics that appeal to their life styles (vegan, organic, etc.), tastes and personal preferences.

### TECHNICAL FIELD

The present invention provides systems, kits and methods for user customizable cosmetics including but not limited to foundation, blush, eye-shadow, lip products, and nail polish.

### BACKGROUND

There are many types of cosmetics available to users. There are seemingly unlimited options, but this is not true for the user's experience. There are a limited number of cosmetics available on the shelves or in stock for the user to readily obtain. They are created by manufacturers, shipped to retailers and eventually provided to the users. Accordingly, the users are limited to the number and choice of colors provided by the manufacturer, and further limited by the retailer. The cosmetic industry has an ever-changing trend focus where end users want individual, customized creations that reflect their personal and artistic tastes. To be truly unique, end-users and cosmetic technicians alike want to differentiate themselves with custom colors and styles often mixing ready-made products, add-ins (such as glitter), resulting in a potentially potent and dangerous mix of chemically laden products. Some creators will remix off-the-shelf products and sell their creations to end users, violating Food and Drug Administration (FDA) requirements. For all of these supply options, the end user is still not aware of how the product was created, and if the ingredients are safe, are derived from animal products, and are aligned with their preferences and life style.

### TECHNICAL PROBLEM

There has been a continuing need to address highly individualized user preferences in the field of cosmetics. Presently, there are no options for achieving individualized cosmetics other than matching by eye, the color that the user desires. There is no solution to address the need for family and friends to experience the artistic joy of creating cosmetics. There is no solution to offer the user the ability to proactively identify, search and filter for ingredients specific to their needs, including but not limited to shimmer, matte, dye-free, vegan-friendly, etc. There is no solution for properly tracking and labeling ingredients during the creation process. There is no solution for giving the user the ability to name their creation, package and label the final product. The present invention is directed to solving the above-discussed problems.

### SUMMARY OF THE INVENTION

Herein are systems, kits and methods for client customizable cosmetics including but not limited to foundation, blush, eye-shadow, lip products, and nail polish. What is created herein, including systems, kits and procedural methods, provides users with an ability to on demand (in the immediate moment), create a custom cosmetic by choosing, for example, colors including textures and light reflective properties, ingredients that align with their artistic and life style preferences, name of the final product to match the intrinsic meaning to the user, etc. The finished product will further be packaged on the spot and be provided a Food and Drug Administration (FDA) compliant label, which displays the ingredients therein for future reference.

The systems, kits and procedural methods of the present invention include a full range of on demand mixing powders and solutions, along with tools for creating, mixing, naming, labeling, and tracking past uses for future reference. The systems, kits and procedural methods can be provided in large/bulk packaging, small container packaging, or in custom, fit to order amounts for individual users. The cosmetic product may be applied immediately after creation, or stored in the packaging for future use. The methods of the invention include setup for commercial/retail or personal use, and include features to maximize the experience of the user when creating the product.

A characteristic feature of the present invention is in the facilitated production of a highly customizable cosmetic product, as these systems, kits and procedural methods allow the end user to create an on demand, custom product.

### DESCRIPTION OF THE EMBODIMENTS

System and/or Kit

Each system and kit can contain one or more of the following:
Instructions (written or verbal, provided with other materials or available online)
Means of measurement to form proper mixing ratios, including scoops, cups, specific bottles or containers of various sizes, etc.
   There is a separate method for making a cream based cosmetic, something with glitter, shimmer, or a product that contains jelly-style, matte, mica or powder, these multiple means of measurement allow for functionality, flexibility and variation.
Cosmetically safe raw materials such as bases (pigmented and suspensions), powders, color concentrations, solvents, film-forming agents, resins, plasticizers, oils, alcohols, etc.
Mixing methods/tools such a hand mixing tools, mixing trays, beads or internal agitators to promote mixing, external vibration and mixing machines that vibrate and mix the cosmetic product while the product is held by the user, vibration and mixing machines that independently support the product, such as through straps, during vibrating and mixing, etc.
Containers such as pans, press pans, individual packaging, scoops, cups, bottles, jars, funnels, gallon or larger size volume storage containers with dispensers, etc.
Applicators such as brushes, droppers, cotton balls, cotton swabs, etc.
Small or craft batch size capabilities for retail users
Color mixing solutions, including:
   Interior agitation devices (i.e. beads)
   External devices including mixers with or without straps or restrictions.
Naming Device for receiving inputs such as letters, numbers, symbols, etc.
Naming Labels with or without adhesive for immediate or later application to bottle.
Placemats, aprons, protective head and face wear for creating a safe user environment.
Ingredient tracking protocol for future reference and re-creation of the product.
Ingredient Label Input Device
Ingredient Label Creator, such as:
   Handheld, on demand printer able to print an adhering label to the packaged product created by user.
   Self-contained on demand printer with the ability to print a custom label without requiring connection to the internet.
Recipe Cards and Formulation Guides
Liability Waivers
Applicator for affixing the name to the packaged creation/product
Database and printed materials searchable by the user to determine product aspects such as dye-free, vegan, organic, etc.
Software that captures ingredient profiles, user inputs, customizable features, and creates outputs to be documented via labels
Kits—which could include several or all of the items listed above.

### General Method for Creating Custom Color

In the following basic flowchart, the steps are performed in the recited order, although one or more steps may be omitted.
1. Selecting a method for ingredient tracking or follow a previously created recipe card.
   a. Note: recipe cards can be paper-based, online and created with an app.
2. Optionally adding alcohol into a small cup or mixing container (e.g. 30 ml volume).
3. Optionally adding at least one cosmetically safe mica, powder or pigment to the small cup.
4. Mixing the product from Steps 2 & 3 using, for example, a small bamboo stir stick (e.g. 3.5 inch) until no dry powder remains.
5. Adding one or more cosmetically safe bases to the small cup.
6. Mix all ingredients from Step 5 using, for example, a small bamboo stick, being careful not to spill.
7. Select a new/unused final packaging bottle.
8. Add the mixture from Step 6 into the final packaging bottle.
9. Add agitators into the final packaging bottle.
10. Insert a brush or other applicator into the final packaging bottle.
11. On top of the applicator added in Step 10, screw on a cap to the final packaging bottle.
   a. In this case, Step 11 will combine and lock the applicator to the cap. However, other applicators and caps allow for different means of locking the applicator to the cap and sealing the final packaging bottle. For instance, where a brush is already assembled with a cap, insert brush with the affixed cap into the final packaging bottle and seal in one step.
12. With the fully sealed final packaging bottle, mix by hand or other manual means, or preferably using an external mixing machine between 5 and 30 seconds. The machine will work manually moving the bottle and by causing the agitators to vibrate within the mixed solution. Mixing time depends on ingredient characteristics.
13. Choose a name for the final product.
14. Type the name into the naming label printer.
15. Print the naming label, which may have an adhesive.
16. Adhere the naming label onto the final packaging bottle.
17. Based on the ingredients used, create a final ingredients label, which may preferably be an FDA compliant label.
18. Print and adhere the final ingredients label to the final packaging bottle or insert the final ingredients label with the final product packaging.
19. File (or refile or save) the ingredients recipe for future reference.

The cosmetically safe mica, powder and pigment may be those known and available to a person having ordinary skill in the art. However, the specific combination of these materials with other cosmetic ingredients to achieve customized cosmetics is novel.

The cosmetically safe bases may be those known and available to a person having ordinary skill in the art. However, the specific combination of these materials with other cosmetic ingredients to achieve customized cosmetics is novel. Preferred cosmetically safe bases include luster bases and glamour bases.

Luster bases include, for example, Butyl Acetate, Ethyl Acetate, Nitrocellulose, Adipic Acid/Neopentyl Glycol/Trimellitic Anhydride Copolymer, Acetyl Tributyl Citrate, Isopropyl Alcohol, Acrylates Copolymer, Stearalkonium Bentonite, N-Butyl Alcohol, Styrene/Acrylates Copolymer, Benzophenone-1, Silica, Alumina, and Trimethylpentanediyl Dibenzoate.

Glamour bases include, for example, Butyl Acetate, Ethyl Acetate, Nitrocellulose , Acetyl Tributyl Citrate, Isobutyl Acetate, Isopropyl Alcohol, Adipic Acid/Neopentyl Glycol/Trimellitic Anhydride Copolymer, Silica, Styrene/Acrylates Copolymer, N-Butyl Alcohol, Benzophenone-1, and D&C Violet #2.

### Method for Creating Custom Color

Basic Flowchart to create “Georgia Pink” nail polish:
1. Select a method for ingredient tracking or follow a previously created recipe card. In this case use a paper-based previously created recipe card.
   a. Note, recipe cards can be paper-based, online and created with an app.
2. Using a dropper, release 4 drops of alcohol into a small cup or mixing container (i.e. 30 ml volume). NOTE: Not all customizations require this first alcohol step, but in this case it is required.
3. Measure 0.6-cc (cubic centimeter) of Rouge Red color powder (mica—Bis (Glycidoxyphenyl) propane/Bisaminomethyl-norbornane Copolymer, Aluminum Hydroxide, Acid Yellow 3 (C.I.47005), Red #28 (C.I.45410), Red #22 (C.I.45380), Red 30 (C.I.77360)) using, for example, a scoop, and add this to the small cup. NOTE: Not all customizations require this first step, but in this case it is required.
4. Hand mix product from Steps 2 & 3 using, for example, a small bamboo stir stick (i.e. 3.5 inch) until no dry powder remains.
5. Add 8 ml of white tinted base and 7 ml of luster base to the small cup.
6. Mix all ingredients from Steps 4 and 5 using, for example, a small bamboo stick, being careful not to spill.
7. Select a new/unused final packaging bottle, in this case a clear, ½ ounce bottle.
8. Insert a funnel into the final packaging bottle, pour the mixture from Step 6 slowly into the funnel and ultimately into the final packaging bottle.
9. Place agitators into the final packaging bottle, in this case two small stainless-steel agitators.
10. Insert a brush or other applicator into the final packaging bottle.
11. On top of the applicator added in Step 10, screw on a cap to the final packaging bottle.
   a. In this case, Step 10 will combine and lock the applicator to the cap. However, other applicators and caps allow for different means of locking the applicator to the cap and sealing the final packaging bottle. For instance, where a brush is already assembled with a cap, insert brush with the affixed cap into the final packaging bottle in one step.
12. With the fully sealed final packaging bottle, mix by hand or other manual means, or ideally using an external mixing machine between 5 and 30 seconds. The machine will work manually moving the bottle and by causing the agitators to vibrate within the mixed solution. Mixing time depends on ingredient characteristics.
13. Choose a name for the final product, in this case “Georgia Pink”.
14. Type the name into the naming label printer.
15. Print the naming label.
16. Place the adhering naming label onto final packaging bottle.
17. Based on ingredients used, consult the tracking document, in this case a recipe card, and combine the ingredients to create a final ingredients label, which may preferably be an FDA compliant label.
18. Print and adhere the final ingredients label to the final packaging bottle or insert the final ingredients label with the final product packaging.
19. File (or refile or save) the ingredients recipe for future reference.

NOTE: The general method above may be modified to use more or less volume of materials, containers, and tools in order to created larger or smaller amounts of the product for storage and future use. For instance, various amounts and types of pigments and bases can be used to create cosmetics of various colors and having various effects.

As is customary with cosmetics, the containers may be glass, plastic of other suitable material and may contain a steel ball bearing to facilitate mixing upon shaking or agitation. Two ball bearings are standard in the industry, although more or less may be used. The finish container may also include a cap with an included applicator. Because kits including the items may include pre-determined mixing instructions and ratios, some embodiments will follow instructions directly, while others will be created by users.

Where the final product is a nail polish, for instance, the kit will contain a base (or variety of bases) depending on kit size and a range of color powder pigments and micas along with glitter add-ins or color concentrates depending on the kit to achieve a desired custom type or color nail polish allowing end users to know and select specific polish types such as dye-free or vegan friendly ingredients. Kits will contain the above variety of ingredients along with instructions, mixing cups, stir sticks, appropriately sized funnels, empty nail polish bottles (with accompanying brushes, caps and agitator mixing balls), ingredient database, end-user MSDS info, custom label maker to allow the end user to creatively name their product and a custom polish mixer. Some kits will additionally include mixing mat(s), apron(s), or recipe cards. Other products created would follow a similar system, kit and procedural method.

The systems, kits and methods recited herein can be performed by an end user in a residential location/setting or a business location/setting such as a salon or studio, or performed by end users with professional guidance such as by a salon or studio professional at a salon setting. The systems, kits and methods herein may also be portable and performed at portable workstations at various venues.

It should be recognized that the invention is not limited to the above described embodiments, but is intended to include all changes and modifications that are within the scope and spirit of the invention. For instance, different combinations, including more or less of the above, can be incorporated by the user. Although it is contemplated herein that a user or professional on behalf of a user may wish to obtain and use the entire range of systems, kits and procedural methods as alluded to above, some embodiments involve the use of a limited number of embodiments to achieve a desired result. In some embodiments, the ingredient containers may have a variety of sizes and types, and the final product containers may have a variety of sizes and types.

What is claimed:

1. A method for producing a custom nail polish, comprising in order:
step 1: optionally adding an alcohol into a mixing container,
step 2: optionally adding at least one cosmetically safe mica, powder or pigment to the mixing container,
step 3: where steps 1 and 2 are performed, mixing the product of step 2 until no dry powder remains,
step 4: adding at least one base into the mixing container,
step 5: mixing the product of step 4 until uniform,
step 6: adding the product of step 5 into a nail polish container,
step 7: adding a cosmetic agitator into the nail polish container,
step 8: sealing the nail polish container by adding a cosmetic applicator and cap,
step 9: mixing the sealed nail polish container for 5 to 30 seconds, wherein the mixing is conducted by manual agitation or with an external mixing machine configured to hold and agitate the sealed nail polish container,
step 10: printing a nail polish container name label,
step 11: affixing the name label to the nail polish container,
step 12: printing an ingredients label for the custom nail polish, and
step 13: affixing the ingredients label to the nail polish container,
wherein the method can be carried out in a residential setting or in a business setting, wherein the business setting is selected from the group consisting of a salon, a studio, and a portable salon workstation.

2. The method according to claim 1, wherein the custom nail polish is Georgia Pink nail polish, comprising:
in step 1, adding 4 drops of alcohol into the mixing container,
in step 2, adding 0.6-cc of Rouge Red mica to the mixing container,
in step 3, mixing the product of step 2 until no dry powder remains,
in step 4, adding 8 ml white tinted base and 7 ml suspension base into the mixing container,
in step 10, printing a name label reciting “Georgia Pink”, and
in step 12, printing an ingredients label reciting the ingredients in steps 1-4,
wherein the Rouge Red mica comprises Bis (Glycidoxyphenyl) propane/Bisaminomethyl-norbornane Copolymer, Aluminum Hydroxide, Acid Yellow 3 (C.I.47005), Red #28 (C.I.45410), Red #22 (C.I.45380), Red 30 (C.I.77360).

3. The method according to claim 1, wherein the custom nail polish is H-town Rep'n nail polish, comprising:
in step 2, adding 0.9-cc of Rouge Red mica to the mixing container,
in step 4, adding 4 ml red tinted base and 11 ml of luster base into the mixing container,
in step 10, printing a name label reciting “H-town Rep'n”, and
in step 12, printing an ingredients label reciting the ingredients in steps 2 and 4,
wherein the Rouge Red mica comprises Bis (Glycidoxyphenyl) propane/Bisaminomethyl-norbornane Copolymer, Aluminum Hydroxide, Acid Yellow 3 (C.I.47005), Red #28 (C.I.45410), Red #22 (C.I.45380), Red 30 (C.I.77360).

4. The method according to claim 1, wherein the custom nail polish is Purple Parrot nail polish, comprising:
in step 2, adding 0.6-cc of Violet mica to the mixing container,
in step 4, adding 7 ml of white tinted base and 10 ml of luster base into the mixing container,
in step 10, printing a name label reciting “Purple Parrot”, and
in step 12, printing an ingredients label reciting the ingredients in steps 2 and 4, wherein the Violet mica comprises Manganese Violet (77742).

5. The method according to claim 1, wherein the custom nail polish is Blue Suede Shoes nail polish, comprising:
in step 1, adding 4 drops of alcohol into a mixing container,
in step 2, adding 0.6-cc of Blue Suede pigment to the mixing container,
in step 3, mixing the product of step 2 until no dry powder remains,
in step 4, adding 1 ml of white tinted base, 12 ml of luster base, and 1 ml of black tint base into the mixing container,
in step 10, printing a name label reciting “Blue Suede Shoes”, and
in step 12, printing an ingredients label reciting the ingredients in steps 1-4,
wherein the Blue Suede pigment comprises Bis (glycidoxyphenyl) propane/Bisaminomethyl-norbornane, Copolymer, Aluminum Hydroxide, Ext. Violet 2 (60730), Red 28 (45410), Red 22 (45380), Ultramarines (77007).

6. The method according to claim 1, wherein the custom nail polish is Tranquility Bay nail polish, comprising:
in step 2, adding 0.75-cc of Blue Pearls mica to the mixing container,
in step 4, adding 2 ml of white tinted base and 15 ml of luster base into the mixing container,
in step 10, printing a name label reciting “Tranquility Bay”, and
in step 12, printing an ingredients label reciting the ingredients in steps 2 and 4,
wherein the Blue Pearls mica comprises Mica (77019), Titanium Dioxide (77891), Ultramarines (77007), Chromium Oxide Green (77288).

7. The method according to claim 1, wherein the custom nail polish is Sea Grass nail polish, comprising:
in step 2, adding 0.75-cc of Spring Mint mica to the mixing container,
in step 4, adding 5 ml of white tinted base, 4 ml of luster base, and 8 ml of glamour base into the mixing container,
in step 10, printing a name label reciting “Sea Grass”, and
in step 12, printing an ingredients label reciting the ingredients in steps 2 and 4,
wherein the Spring Mint mica comprises Mica (77019), Titanium Dioxide (77891), Tin Oxide (778161), Chromium Oxide Green (77288).

8. The method according to claim 1, wherein the custom nail polish is Key Lime Pie nail polish, comprising:
in step 1, adding 2 drops of alcohol into a mixing container,
in step 2, adding 0.15-cc of Lakeview pigment and 1.5-cc of Silver White mica to the mixing container,
in step 3, mixing the product of step 2 until no dry powder remains,
in step 4, adding 5 ml of white tinted base, 10 ml of luster base, and 1 drop of sunny liquid concentrate into the mixing container,
in step 10, printing a name label reciting “Key Lime Pie”, and
in step 12, printing an ingredients label reciting the ingredients in steps 1-4,
wherein the Lakeview pigment comprises Bis (glycidoxyphenyl) propane/Bisaminomethyl-norbornane, Copolymer, Aluminum Hydroxide, Ext. Violet 2 (60730), Red 28 (45410), Red 22 (45380), Ultramarines (77007), Black 2 (77266), and
wherein the Silver White mica comprises Mica (77019), Titanium Dioxide (77891).

9. The method according to claim 1, wherein the custom nail polish is Gipsy Jazz nail polish, comprising:
in step 2, adding 0.75-cc of Slate Blue mica to the mixing container,
in step 4, adding 3 ml of black tinted base and 10 ml of glamour base into the mixing container,
in step 10, printing a name label reciting “Gipsy Jazz”, and
in step 12, printing an ingredients label reciting the ingredients in steps 2 and 4,
wherein the Slate Blue mica comprises Talc, Mica (77019), Zinc Stearate, optionally Titanium Dioxide (77891), Iron Oxide (77491), Ferric Ferrocyanide (77510), Ultramarines (77007), Bismuth Oxychloride (77163).

10. The method according to claim 1, wherein the custom nail polish is Heavy Metal nail polish, comprising:
in step 2, adding 0.75-cc of Summer Storm mica to the mixing container,
in step 4, adding 4 ml of black tinted base and 13 ml of luster base into the mixing container,
in step 10, printing a name label reciting “Heavy Metal”, and
in step 12, printing an ingredients label reciting the ingredients in steps 2 and 4,
wherein the Summer Storm mica comprises Mica (77019), Titanium Dioxide (77891), Iron Oxide (77491), Boron Nitride.

11. The method according to claim 1, wherein the custom nail polish is Sultry Soul nail polish, comprising:
in step 4, adding 5 ml of black tinted base, 5 ml of white tinted base, 2.5 ml of luster base, and 3.5 ml of glamour base into the mixing container,
in step 10, printing a name label reciting “Sultry Soul”, and
in step 12, printing an ingredients label reciting the ingredients in step 4.

12. The method according to claim 1, wherein the custom nail polish is Hokie Stone nail polish, comprising:
in step 2, adding 0.75-cc of Nude Gray to the mixing container,
in step 4, adding 5 ml of white tinted base and 10 ml of luster base into the mixing container,
in step 10, printing a name label reciting “Hokie Stone”, and
in step 12, printing an ingredients label reciting the ingredients in steps 2 and 4, wherein the Nude Gray is illite (purifying clay).

13. The method according to claim 1, wherein the custom nail polish is Heart of Gold nail polish, comprising:
in step 2, adding 0.75-cc of Light Brown Sugar mica and 0.15-cc of Underwater Gold mica to the mixing container,
in step 4, adding 1 ml of white tinted base and 15 ml of luster base into the mixing container,
in step 10, printing a name label reciting “Heart of Gold”, and
in step 12, printing an ingredients label reciting the ingredients in steps 2 and 4,
wherein the Light Brown Sugar mica comprises Mica (77019), Titanium Dioxide (77891), Iron Oxide (77491), Iron Oxide (77499), (Tin Oxide (778161), and
wherein the Underwater Gold mica comprises Mica (77019), Titanium Dioxide (77891), Iron Oxide (77491).

14. The method according to claim 1, wherein the custom nail polish is Fiery Funk nail polish, comprising:
in step 2, adding 0.45-cc of Strong Drama mica and 0.15-cc of pink terracotta mica to the mixing container,
in step 4, adding 1 ml of red tinted base, 5 ml of luster base, 7 ml of glamour base, and 2 ml of black tinted base into the mixing container,
in step 10, printing a name label reciting “Fiery Funk”, and
in step 12, printing an ingredients label reciting the ingredients in steps 2 and 4,
wherein the Strong Drama mica comprises Mica (77019), Iron Oxide (77491), and
wherein the pink terracotta mica comprises Mica (77019), Talc, Zinc Stearate, optionally Titanium Dioxide (77891), Iron Oxide (77491).

15. The method according to claim 1, wherein the custom nail polish is Emerald Envy nail polish, comprising:
in step 2, adding 0.45-cc of Gemstone Green mica and 0.3-cc of Silver White mica to the mixing container,
in step 4, adding 3 ml of white tinted base and 15 ml of glamour base into the mixing container,
in step 10, printing a name label reciting “Emerald Envy”, and
in step 12, printing an ingredients label reciting the ingredients in steps 2 and 4,
wherein the Gemstone Green mica comprises Mica (77019), Titanium Dioxide (77891), Iron Oxide (77491), Chromium Oxide Green (77288), and
wherein the Silver White mica comprises Mica (77019), Titanium Dioxide (77891).

16. The method according to claim 1, wherein the custom nail polish is Chameleon Club nail polish, comprising:
in step 2, adding 0.9-cc of Changing Times mica to the mixing container,
in step 4, adding 15 ml of glamour base into the mixing container,
in step 10, printing a name label reciting “Chameleon Club”, and
in step 12, printing an ingredients label reciting the ingredients in steps 2 and 4,
wherein the Changing Times mica comprises Mica (77019), Iron Oxide (77491).

17. The method according to claim 1, wherein the custom nail polish is Lovely Lolo nail polish, comprising:
in step 2, adding 0.75-cc of Pink Angel Wings mica to the mixing container,
in step 4, adding 15 ml of glamour base into the mixing container,
in step 10, printing a name label reciting “Lovely Lolo”, and
in step 12, printing an ingredients label reciting the ingredients in steps 2 and 4,
wherein the Pink Angel Wings mica comprises Mica (77019), Titanium Dioxide (77891), Iron Oxide (77499), Carmine (75470).

18. The method according to claim 1, wherein the custom nail polish is Gimme All Ur Lov'n nail polish, comprising:
in step 2, adding 0.6-cc of Neutral Pink Shimmer mica to the mixing container,
in step 4, adding 15 ml of luster base and 1 ml of black tinted base into the mixing container,
in step 10, printing a name label reciting “Gimme All Ur Lov'n”, and
in step 12, printing an ingredients label reciting the ingredients in steps 2 and 4,
wherein the Neutral Pink Shimmer mica comprises Mica (77019), Titanium Dioxide (77891), Iron Oxide (77491), Carmine (75470), Boron Nitride.

19. The method according to claim 1, wherein the custom nail polish is Always A Party nail polish, comprising:
in step 2, adding 0.75-cc of Sassy Granny mica to the mixing container,
in step 4, adding 15 ml of glamour base and 1 ml of white tinted base into the mixing container,
in step 10, printing a name label reciting “Always A Party”, and
in step 12, printing an ingredients label reciting the ingredients in steps 2 and 4,
wherein the Sassy Granny mica comprises Mica (770179), Titanium Dioxide (77891), Tin Oxide (778161), Iron Oxide (77491).

20. The method according to claim 1, wherein the custom nail polish is Wild Pink Flamingo nail polish, comprising:
in step 2, adding 1.05-cc of 5^{th} Avenue Pink pigment to the mixing container,
in step 4, adding 7.5 ml of white tinted base, 7.5 ml of luster base, and 1 ml of black tinted base into the mixing container,
in step 10, printing a name label reciting “Wild Pink Flamingo”, and
in step 12, printing an ingredients label reciting the ingredients in steps 2 and 4,
wherein the 5^{th} Avenue Pink pigment comprises Bis (glycidoxyphenyl) propane/Bisaminomethyl-norbornane, Copolymer, Aluminum Hydroxide, Ext. Violet 2 (60730), Red 28 (45410), Red 22 (45380), Ultramarines (77007).

21. The method according to claim 1, wherein the custom nail polish is She's a Pip, Pearl nail polish, comprising:
in step 2, adding 0.6-cc of White Velvet mica to the mixing container,
in step 4, adding 3 ml of white tinted base and 14 ml of luster base into the mixing container,
in step 10, printing a name label reciting “She's a Pip, Pearl”, and
in step 12, printing an ingredients label reciting the ingredients in steps 2 and 4,
wherein the White Velvet mica comprises Mica (77019), Titanium Dioxide (77891).

22. The method according to claim 1, wherein the custom nail polish is Peggy's Sweet Tea nail polish, comprising:
in step 2, adding 0.75-cc of Sassy Granny mica to the mixing container,
in step 4, adding 16 ml of luster base into the mixing container,
in step 10, printing a name label reciting “Peggy's Sweet Tea”, and
in step 12, printing an ingredients label reciting the ingredients in steps 2 and 4,
wherein the Sassy Granny mica comprises Mica (770179), Titanium Dioxide (77891), Tin Oxide (778161), Iron Oxide (77491).

23. The method according to claim 1, wherein the custom nail polish is East Texas Watermelon nail polish, comprising:
in step 2, adding 0.75-cc of Bright Coral mica to the mixing container,
in step 4, adding 3 ml of white tinted base, 10 ml of luster base, and 5 ml of glamour base into the mixing container,
in step 10, printing a name label reciting “East Texas Watermelon”, and
in step 12, printing an ingredients label reciting the ingredients in steps 2 and 4,
wherein the Bright Coral mica comprises Mica (77019), Titanium Dioxide (77891), Tin Oxide (778161), Red #40 (16035:1), Red #6 (15850).

24. A customizable cosmetic kit, comprising:
a means for measuring a cosmetic raw material,
a cosmetic raw material, which is at least one selected from the group consisting of a pigment, a suspension, a powder, a solvent, a film-forming agent, a resin, a plasticizer, an oil, an alcohol, glitter, and mica,
a means for mixing the raw material,
a container configured to hold a cosmetic,
a cosmetic agitator configured to fit inside the container,
a cosmetic applicator,
a polishing element, and
optionally:
a container filling tool,
at least one selected from the group consisting of a placemat, an apron, a protective head cover, and a protective face cover,
an instruction manual,
a container labeling device
a container label,
a label applicator, and
a recipe card.
